(19) United States
(12) Patent Application Publication
(10) Pub. No.: US 2022/0072123 A1
(43) Pub. Date: Mar. 10, 2022

(54) METHODS OF INTRODUCING FOOD ALLERGENS

(71) Applicant: BEFORE BRANDS, Inc., Menlo Park, CA (US)

(72) Inventors: Christopher Cornyn, Woodside, CA (US); Ashley Dombkowski, Woodside, CA (US)

(21) Appl. No.: 17/422,222

(22) PCT Filed: Jan. 10, 2020
(86) PCT No.: PCT/US2020/013161

§ 371 Date: Jul. 12, 2021

Related U.S. Application Data
(60) Provisional application No. 62/790,699, filed on Jan. 10, 2019.

(51) Int. Cl. A61K 39/35

(52) U.S. Cl. CPC A61K 39/35; A61K 2039/542

(57) ABSTRACT

Methods for introducing allergens to a human subject are provided.

### CROSS-REFERENCE TO RELATED APPLICATION

This application claims priority to U.S. Provisional Patent Application No. 62/790,699, filed Jan. 10, 2019, the entire contents of which are incorporated by reference herein in their entirety.

### BACKGROUND

Allergy is a disorder of the immune system characterized by the occurrence of allergic reactions to normally harmless environmental substances. Allergies are caused by allergens, which may be present in a wide variety of sources, including but not limited to pollens or other plant components, dust, molds or fungi, foods, additives, latex, transfusion reactions, animal or bird danders, insect venoms, radiocontrast medium, medications or chemicals. Common allergic reactions include eczema, hives, hay fever, asthma, and reactions to venoms. Mild allergies like hay fever are highly prevalent in the human population and cause symptoms such as allergic conjunctivitis, itchiness, and runny nose. In some people, severe allergies to environmental or dietary allergens or to medication may result in life-threatening anaphylactic reactions and potentially death, if left untreated.

A food allergy is an adverse immune response to a food allergen, e.g., a food protein. Common food allergens are found in shellfish, peanuts, tree nuts, fish, milk, eggs, soy and fresh fruits such as strawberries, mangoes, bananas, and apples. Immunoglobulin E (IgE)-mediated food allergies are classified as type-I immediate hypersensitivity reactions. These allergic reactions have an acute onset (from seconds to one hour) and the accompanying symptoms may include angioedema (soft tissue swelling of the eyelids, face, lips, tongue, larynx and trachea), hives, itching of the mouth, throat, eyes, or skin, gastrointestinal symptoms such as nausea, vomiting, diarrhea, stomach cramps, or abdominal pain, rhinorrhea or nasal congestion, wheezing, shortness of breath, or difficulty swallowing, and even anaphylaxis, a severe, whole-body allergic reaction that can result in death. It is estimated that 1 out of 12 children under 21 years of age have a doctor's diagnosis of food allergies, and over $24 billion is spent per year on health care costs for food allergic reactions, largely due to about 90,000 visits to the ER per year in the U.S. due to food induced anaphylactic symptoms. Moreover, there are still deaths that occur every year due to fatal food allergies.

Accordingly, there exists a need in the art for compositions that can prevent and/or treat allergies, and methods for introducing allergens to a human subject to prevent and/or treat allergies

### SUMMARY

The disclosure is directed, at least in part, to methods for introducing allergens to a human subject and methods of treating or preventing food allergies in a human subject.

Provided herein, in an embodiment, is a method for introducing allergens to a human subject (and/or treating or preventing food allergies in a human subject) comprising administering to the subject a first allergenic unit and optionally administering to the subject a second, third, fourth, fifth, sixth, seventh, eighth, and/or ninth allergenic unit, wherein each successive allergenic unit is administered at least about 2 days after the previous allergenic unit is administered.

Contemplated allergenic units comprise, e.g., peanut allergen, soy allergen, egg allergen, milk allergen, almond allergen, cashew allergen, walnut allergen, pecan allergen, hazelnut allergen, pistachio allergen, salmon allergen, cod allergen, shrimp allergen, sesame allergen, wheat allergen oat allergen, and/or Vitamin D. In embodiments, an allergenic unit comprises one or more tree nut allergens, e.g., an almond allergen, cashew allergen, walnut allergen, pecan allergen, hazelnut allergen, and/or pistachio allergen. In embodiments, an allergenic unit comprises one or more fish allergens, e.g., salmon allergen and/or cod allergen. In embodiments, an allergenic unit comprises one or more shellfish allergens, e.g., shrimp allergen. In embodiments, allergenic units comprise 30 mg of each allergen present.

For example, a contemplated method comprises administering to a subject a first allergenic unit (e.g., an allergenic unit comprising about 30 mg of peanut allergen and optionally Vitamin D) wherein the first allergenic unit is administered daily, every other day, every 2 or 3 days, or weekly. A contemplated method further comprises administering to the subject a second allergenic unit (e.g., an allergenic unit comprising about 30 mg of soy allergen and optionally Vitamin D), wherein the second allergenic unit is administered at least about 2 days after the first allergenic unit is first administered, and wherein the second allergenic unit is administered daily, every other day, every 2 or 3 days or weekly. A contemplated method further comprises administering to the subject a third allergenic unit (e.g., an allergenic unit comprising about 30 mg of egg allergen and optionally Vitamin D), wherein the third allergenic unit is administered at least about 2 days after the second allergenic unit is first administered, and wherein the third allergenic unit is administered daily, every other day, every 2 or 3 days or weekly. A contemplated method further comprises administering to the subject a fourth allergenic unit (e.g., an allergenic unit comprising about 30 mg of milk allergen and optionally Vitamin D), wherein the fourth allergenic unit is administered at least about 2 days after the third allergenic unit is first administered, and wherein the fourth allergenic unit is administered daily, every other day, every 2 or 3 days or weekly. A contemplated method further comprises administering to the subject a fifth allergenic unit (e.g., an allergenic unit comprising about 30 mg of almond allergen, cashew allergen, walnut allergen, pecan allergen, hazelnut allergen, pistachio allergen, or any combination thereof, and optionally Vitamin D), wherein the fifth allergenic unit is administered at least about 2 days after the fourth allergenic unit is first administered, and wherein the fifth allergenic unit is administered daily, every other day, every 2 or 3 days or weekly. A contemplated method further comprises administering to the subject a sixth allergenic unit (e.g., an allergenic unit comprising about 30 mg of salmon allergen, cod allergen, or any combination thereof, and optionally Vitamin D) wherein the sixth allergenic unit is administered at least about 2 days after the fifth allergenic unit is first administered, and wherein the sixth allergenic unit is administered daily, every other day, every 2 or 3 days or weekly. A contemplated method further comprises administering to the subject a seventh allergenic unit (e.g., an allergenic unit comprising about 30 mg of shrimp allergen and optionally Vitamin D), wherein the seventh allergenic unit is administered at least about 2 days after the sixth allergenic unit is first administered, and wherein the seventh allergenic unit is administered daily, every other day, every 2 or 3 days or weekly. A contemplated method further comprises administering to the subject an eighth allergenic unit (e.g., an allergenic unit comprising about 30 mg of sesame allergen and optionally Vitamin D) wherein the eighth allergenic unit is administered at least about 2 days after the seventh allergenic unit is first administered, and wherein the eighth allergenic unit is administered daily, every other day, every 2 or 3 days or weekly. A contemplated method further comprises administering to the subject a ninth allergenic unit (e.g., an allergenic unit comprising about 30 mg of wheat allergen, oat allergen, or any combination thereof, and optionally Vitamin D), wherein the ninth allergenic unit is administered at least about 2 days after the eighth allergenic unit is first administered, and wherein the ninth allergenic unit is administered daily, every other day, every 2 or 3 days or weekly.

For example, a contemplated method comprises a first allergenic unit comprising about 30 mg of peanut allergen and optionally Vitamin D; a second allergenic unit comprising about 30 mg of soy allergen, wherein the second allergenic unit is administered at least about 2 days after the first allergenic unit is first administered, a third allergenic unit comprising about 30 mg of egg allergen, wherein the third allergenic unit is administered at least about 2 days after the second allergenic unit is first administered; a fourth allergenic unit comprising about 30 mg of milk allergen, wherein the fourth allergenic unit is administered at least about 2 days after the third allergenic unit is first administered; a fifth allergenic unit comprising about 30 mg each of almond allergen, cashew allergen, walnut allergen, pecan allergen, hazelnut allergen, and pistachio allergen, wherein the fifth allergenic unit is administered at least about 2 days after the fourth allergenic unit is first administered; a sixth allergenic unit comprising about 30 mg each of salmon allergen and cod allergen, wherein the sixth allergenic unit is administered at least about 2 days after the fifth allergenic unit is first administered; a seventh allergenic unit comprising about 30 mg of shrimp allergen, wherein the seventh allergenic unit is administered at least about 2 days after the sixth allergenic unit is first administered; an eighth allergenic unit comprising about 30 mg of sesame allergen, wherein the eighth allergenic unit is administered at least about 2 days after the seventh allergenic unit is first administered; a ninth allergenic unit comprising about 30 mg each of wheat allergen and oat allergen, wherein the ninth allergenic unit is administered at least about 2 days after the eighth allergenic unit is first administered.

Also provided herein, in an embodiment, is a method for introducing food allergens to a human subject for the treatment or prevention of food allergies, comprising administering to the subject two or more allergenic units, wherein the initial administration of each allergenic unit is staggered, each allergenic unit is administered daily, every other day, every 2 or 3 days, or weekly, the initial administration of each additional allergenic unit is at least two days after the initial administration of a previously administered allergenic unit, and each previously administered allergenic unit continues to be administered in addition to each additional allergenic unit as each additional allergenic unit is administered. In certain embodiments, each allergenic unit comprises a different food allergen selected from the group consisting of: an allergenic unit comprising least one tree nut selected from the group consisting of almond, cashew, walnut, pecan, hazelnut, and pistachio; an allergenic unit comprising a white fish and/or a pink fish; and an allergenic unit comprising shellfish.

Also provided herein, in an embodiment, is a method for introducing food allergens to a human subject for the treatment or prevention of food allergies, comprising administering to the subject two allergenic units with a staggered start of administration, wherein: a first allergenic unit is administered daily, every other day, every 2 or 3 days, or weekly; a second allergenic unit is administered daily, every other day, every 2 or 3 days, or weekly, the start of the administration of the second allergenic unit is at least two days after the start of the administration of the first allergenic unit, and the administration of the first allergenic unit continues during the administration of the second allergenic unit. In certain embodiments, a contemplated method comprises administering to a subject additional allergenic units, wherein each additional allergenic unit is administered daily, every other day, every 2 or 3 days, or weekly, administration of each additional allergenic unit is started at least two days after the start of the administration of the previously added allergenic unit, and the administration of the previously added allergenic unit allergenic unit continues during the administration of each additional allergenic unit. In certain embodiments, each allergenic unit comprises a different food allergen selected from the group consisting of: an allergenic unit comprising least one tree nut selected from the group consisting of almond, cashew, walnut, pecan, hazelnut, and pistachio; an allergenic unit comprising a white fish and/or a pink fish; and an allergenic unit comprising shellfish.

Also provided herein, in an embodiment, is a method for introducing food allergens to a human subject for the treatment or prevention of food allergies, comprising administering to the subject two or more allergenic units, wherein the start of administering each allergenic unit is staggered, and comprises administering a first allergenic unit administered daily, every other day, every 2 or 3 days, or weekly, and administration of each additional allergenic unit is started after at least two days of a previously selected allergenic unit, and wherein the previously selected allergenic unit is continued to be administered in addition to the additional allergenic unit, as each additional unit is administered. In certain embodiments, each allergenic unit is a different food allergen and is selected from the group consisting of: an allergenic unit comprising at least one tree nut selected from the group consisting of almond, cashew, walnut, pecan, hazelnut, and pistachio; an allergenic unit comprising a white fish and/or a pink fish; and an allergenic unit comprising a shellfish.

Also provided herein, in an embodiment, is a method for introducing allergens to a human subject (and/or treating or preventing food allergies in a human subject) comprising administering to the subject two or more allergenic units, wherein a first allergenic unit is administered daily and each additional allergenic unit is administered sequentially; wherein each allergenic unit is selected from the group consisting of: an allergenic unit comprising peanut allergen and optionally Vitamin D; an allergenic unit comprising soy allergen; an allergenic unit comprising egg allergen; an allergenic unit comprising milk allergen; an allergenic unit comprising at least one tree nut allergen selected from the group consisting of almond allergen, cashew allergen, walnut allergen, pecan allergen, hazelnut allergen, and pistachio allergen; an allergenic unit comprising salmon allergen, and cod allergen; an allergenic unit comprising shrimp allergen; an allergenic unit comprising sesame allergen; and an allergenic unit comprising at least one of wheat allergen and oat allergen; wherein each selected allergenic unit is administered after at least two days of administration of a previously selected allergenic unit. In embodiments, allergenic units comprise about 30 mg of each allergen present.

### DETAILED DESCRIPTION

Provided herein are methods for introducing allergens to a human subject and/or treating or preventing food allergies in a human subject. Provided methods may comprise, e.g., administration of a first allergen to a subject followed by the subsequent administration of one or more additional allergens (e.g., a second allergen, a third allergen, etc.) to a subject. Such titrated introduction may be useful in, e.g., avoiding a serious allergic reaction while still providing resistance to allergenic response.

A disclosed method may comprise, e.g., administering to a subject a first allergenic unit and optionally administering to the subject a second, third, fourth, fifth, sixth, seventh, eighth, ninth, tenth, eleventh, twelfth, thirteenth, fourteenth, fifteenth, sixteenth, seventeenth, eighteenth, nineteenth and/or twentieth allergenic unit, wherein each successive allergenic unit is administered after the previous allergenic unit is administered. In embodiments, a disclosed method comprises administering to a subject a first, second, third, fourth, fifth, sixth, seventh, eighth, and ninth allergenic unit, wherein each successive allergenic unit is administered after the previous allergenic unit is administered.

In embodiments, each allergenic unit is administered daily, every other day, every 2, 3, 4, 5, 6, or 7 days, or weekly. In embodiments, each allergenic unit is administered daily, every other day, every 2 or 3 days, or weekly.

In embodiments, each successive allergenic unit is administered at least about 1, 2, 3, 4, 5, 6, 7, 8, 9 or 10 days after the previous allergenic unit is administered. In embodiments, each successive allergenic unit is administered at least about 1, 2, 3, or 4 weeks after the previous allergenic unit is administered. In embodiments, each successive allergenic unit is administered at least about 2 days after the previous allergenic unit is administered.

Each allergenic unit may include an individual allergenic component (e.g., about 1 mg to about 1000 mg, e.g., about 2 mg to about 100 mg, about 5 mg to about 50 mg, about 15 mg, about 20 mg, about 25 mg, or about 30 mg by protein weight of peanut, tree nut, cow's milk, soy, egg, fish or shellfish), or multiple allergenic components (e.g., about 1 mg to about 1000 mg, e.g., about 2 mg to about 100 mg, about 5 mg to about 50 mg, about 15 mg, about 20 mg, about 25 mg, or about 30 mg by protein weight of two or more of peanut, tree nut, cow's milk, soy, egg, fish or shellfish).

For example, a contemplated method comprises administering to a subject a first allergenic unit (e.g., an allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D) wherein the first allergenic unit is administered daily, every other day, every 2 or 3 days, or weekly. In embodiments, a first allergenic unit may include about 30 mg, by protein weight, of peanut, and optionally Vitamin D.

Such contemplated methods may further include administering to the subject a second allergenic unit (e.g., a second allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the second allergenic unit includes a different (e.g., additional) allergenic component from the first allergenic unit. The second allergenic unit, e.g., is administered at least about 1 day after the first allergenic unit is administered (e.g., at least about 2 days, e.g., 2 days, 3 days or 1 week after the first allergenic unit is first administered), and, e.g., is administered daily, every other day, every 2 or 3 days or weekly. In embodiments, a second allergenic unit may include about 30 mg, by protein weight, of soy, and optionally Vitamin D. In embodiments, a second allergenic may include about 30 mg each, by protein weight, of peanut and soy, and optionally Vitamin D.

Such contemplated methods may further include administering to the subject a third allergenic unit (e.g., a third allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the third allergenic unit includes a different (e.g., additional) allergenic component from the second allergenic unit. The third allergenic unit, e.g., is administered at least about 1 day after the second unit is administered (e.g., at least about 2 days, e.g., 2 days, 3 days or 1 week after the second allergenic unit is first administered), and, e.g., is administered daily, every other day, every 2 or 3 days or weekly. In embodiments, a third allergenic unit may include about 30 mg, by protein weight, of egg, and optionally Vitamin D. In embodiments, a third allergenic unit may include about 30 mg each, by protein weight, of peanut, soy and egg, and optionally Vitamin D.

Such contemplated methods may further include administering to the subject a fourth allergenic unit (e.g., a fourth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the fourth allergenic unit includes a different (e.g., additional) allergenic component from the third allergenic unit. The fourth allergenic unit, e.g., is administered at least about 1 day after the third unit is administered (e.g., at least about 2 days, e.g., 2 days, 3 days or 1 week after the third allergenic unit is first administered), and, e.g., is administered daily, every other day, every 2 or 3 days or weekly. In embodiments, a fourth allergenic unit may include about 30 mg, by protein weight, of milk, and optionally Vitamin D. In embodiments, a fourth allergenic unit may include about 30 mg each, by protein weight, of peanut, soy, egg and milk, and optionally Vitamin D.

Such contemplated methods may further include administering to the subject a fifth allergenic unit (e.g., a fifth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the fifth allergenic unit includes a different (e.g., additional) allergenic component from the fourth allergenic unit. The fifth allergenic unit, e.g., is administered at least about 1 day after the fourth unit is administered (e.g., at least about 2 days, e.g., 2 days, 3 days or 1 week after the fourth allergenic unit is first administered), and, e.g., is administered daily, every other day, every 2 or 3 days or weekly. In embodiments, a fifth allergenic unit may include about 30 mg, by protein weight, of a tree nut selected from the group consisting of almond, cashew, walnut, pecan, hazelnut, pistachio, or any combination thereof, and optionally Vitamin D. In embodiments, a fifth allergenic unit may include about 30 mg each, by protein weight, of peanut, soy, egg, milk, and a tree nut selected from the group consisting of almond, cashew, walnut, pecan, hazelnut, pistachio, or any combination thereof, and optionally Vitamin D.

Such contemplated methods may further include administering to the subject a sixth allergenic unit (e.g., a sixth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the sixth allergenic unit includes a different (e.g., additional) allergenic component from the fifth allergenic unit. The sixth allergenic unit, e.g., is administered at least about 1 day after the fifth unit is administered (e.g., at least about 2 days, e.g., 2 days, 3 days or 1 week after the fifth allergenic unit is first administered), and, e.g., is administered daily, every other day, every 2 or 3 days or weekly. In embodiments, a sixth allergenic unit may include about 30 mg, by protein weight, of a fish selected from the group consisting of salmon, cod, or any combination thereof, and optionally Vitamin D. In embodiments, a sixth allergenic unit may include about 30 mg each, by protein weight, of peanut, soy, egg, milk, a tree nut selected from the group consisting of almond, cashew, walnut, pecan, hazelnut, pistachio, or any combination thereof, and a fish selected from the group consisting of salmon, cod, or any combination thereof, and optionally Vitamin D.

Such contemplated methods may further include administering to the subject a seventh allergenic unit (e.g., a seventh allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the seventh allergenic unit includes a different (e.g., additional) allergenic component from the sixth allergenic unit. The seventh allergenic unit, e.g., is administered at least about 1 day after the sixth unit is administered (e.g., at least about 2 days, e.g., 2 days, 3 days or 1 week after the sixth allergenic unit is first administered), and, e.g., is administered daily, every other day, every 2 or 3 days or weekly. In embodiments, a seventh allergenic unit may include about 30 mg, by protein weight, of shrimp, and optionally Vitamin D. In embodiments, a seventh allergenic unit may include about 30 mg each, by protein weight, of peanut, soy, egg, milk, a tree nut selected from the group consisting of almond, cashew, walnut, pecan, hazelnut, pistachio, or any combination thereof, a fish selected from the group consisting of salmon, cod, or any combination thereof, and shrimp, and optionally Vitamin D.

Such contemplated methods may further include administering to the subject an eighth allergenic unit (e.g., an eighth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the eighth allergenic unit includes a different (e.g., additional) allergenic component from the seventh allergenic unit. The eighth allergenic unit, e.g., is administered at least about 1 day after the seventh unit is administered (e.g., at least about 2 days, e.g., 2 days, 3 days or 1 week after the seventh allergenic unit is first administered), and, e.g., is administered daily, every other day, every 2 or 3 days or weekly. In embodiments, an eighth allergenic unit may include about 30 mg, by protein weight, of sesame, and optionally Vitamin D. In embodiments, an eighth allergenic unit may include about 30 mg each, by protein weight, of peanut flour, soy, egg, milk, a tree nut selected from the group consisting of almond, cashew, walnut, pecan, hazelnut, pistachio, or any combination thereof, a fish selected from the group consisting of salmon, cod, or any combination thereof, shrimp, and sesame, and optionally Vitamin D.

Such contemplated methods may further include administering to the subject an ninth allergenic unit (e.g., a ninth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the ninth allergenic unit includes a different (e.g., additional) allergenic component from the eighth allergenic unit. The ninth allergenic unit, e.g., is administered at least about 1 day after the eighth unit is administered (e.g., at least about 2 days, e.g., 2 days, 3 days or 1 week after the eighth allergenic unit is first administered), and, e.g., is administered daily, every other day, every 2 or 3 days or weekly. In embodiments, a ninth allergenic unit may include about 30 mg, by protein weight, of a non-nut plant selected from the group consisting of wheat, oat, or any combination thereof, and optionally Vitamin D. In embodiments, a ninth allergenic unit may include about 30 mg each, by protein weight, of peanut, soy, egg, milk, a tree nut selected from the group consisting of almond, cashew, walnut, pecan, hazelnut, pistachio, or any combination thereof, a fish selected from the group consisting of salmon, cod, or any combination thereof, shrimp, sesame, and a non-nut plant selected from the group consisting of wheat, oat, or any combination thereof, and optionally Vitamin D.

Also provided herein are kits for use in introducing allergens to a human subject. Provided kits may comprise, e.g., a first unit including an allergenic component and an additional unit (e.g., a second unit, a third unit, etc.) wherein the additional unit includes an allergenic component that is different than the first unit. For example, a contemplated kit includes a first unit comprising one allergenic component (e.g., about 1 mg to about 1000 mg, e.g., about 2 mg to about 100 mg, about 5 mg to about 50 mg, about 15 mg, about 20 mg, about 25 mg, or about 30 mg by protein weight of peanut, tree nut, cow's milk, soy, egg, fish or shellfish), and a second unit including a first subunit and a second subunit, wherein the first subunit includes the same allergenic component as the first unit, and the second subunit comprises one allergenic component (e.g., about 1 mg to about 1000 mg, e.g., about 2 mg to about 100 mg, about 5 mg to about 50 mg, about 15 mg, about 20 mg, about 25 mg, or about 30 mg by protein weight of peanut, tree nut, cow's milk, soy, egg, fish or shellfish), wherein the second subunit allergenic component is different than the first subunit allergenic component; and wherein the first subunit and second subunit may be separate or combined.

A disclosed kit may comprise, e.g., a first unit and optionally a second, third, fourth, fifth, sixth, seventh, eighth, ninth, tenth, eleventh, twelfth, thirteenth, fourteenth, fifteenth, sixteenth, seventeenth, eighteenth, nineteenth and/or twentieth unit. Each additional unit may comprise, e.g., a first additional subunit and a second additional subunit, wherein the first additional subunit comprises the same allergenic components as a previous unit, and the second additional subunit comprises one allergenic component (e.g., about 1 mg to about 1000 mg, e.g., about 2 mg to about 100 mg, about 5 mg to about 50 mg, about 15 mg, about 20 mg, about 25 mg, or about 30 mg by protein weight of peanut, tree nut, cow's milk, soy, egg, fish or shellfish), wherein the second additional subunit allergenic component is different than any of the first additional subunit allergenic components; and wherein the first additional subunit and second additional subunit may be separate or combined.

Each unit may include an individual allergenic component (e.g., about 1 mg to about 1000 mg, e.g., about 2 mg to about 100 mg, about 5 mg to about 50 mg, about 15 mg, about 20 mg, about 25 mg, or about 30 mg by protein weight of peanut, tree nut, cow's milk, soy, egg, fish or shellfish), or multiple allergenic components (e.g., about 1 mg to about 1000 mg, e.g., about 2 mg to about 100 mg, about 5 mg to about 50 mg, about 15 mg, about 20 mg, about 25 mg, or about 30 mg by protein weight of two or more of peanut, tree nut, cow's milk, soy, egg, fish or shellfish).

For example, a contemplated kit includes a first unit including one allergenic component (e.g., an allergenic component comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D). In embodiments, a first unit may include about 30 mg, by protein weight, of peanut, and optionally Vitamin D.

Such contemplated kits may further include a second unit (e.g., a second allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the second unit includes a different (e.g., additional) allergenic component from the first unit. In embodiments, a second unit may include about 30 mg, by protein weight, of soy, and optionally Vitamin D. In embodiments, a second unit may include about 30 mg each, by protein weight, of peanut and soy, and optionally Vitamin D.

Such contemplated kits may further include a third unit (e.g., a third allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the third unit includes a different (e.g., additional) allergenic component from the second unit. In embodiments, a third unit may include about 30 mg, by protein weight, of egg, and optionally Vitamin D. In embodiments, a third unit may include about 30 mg each, by protein weight, of peanut, soy and egg, and optionally Vitamin D.

Such contemplated kits may further include a fourth unit (e.g., a fourth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the fourth unit includes a different (e.g., additional) allergenic component from the third unit. In embodiments, a fourth unit may include about 30 mg, by protein weight, of milk, and optionally Vitamin D. In embodiments, a fourth allergenic unit may include about 30 mg each, by protein weight, of peanut, soy, egg and milk, and optionally

Vitamin D.

Such contemplated kits may further include a fifth unit (e.g., a fifth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the fifth unit includes a different (e.g., additional) allergenic component from the fourth unit. In embodiments, a fifth unit may include about 30 mg, by protein weight, of a tree nut selected from the group consisting of almond, cashew, walnut, pecan, hazelnut, pistachio, or any combination thereof, and optionally Vitamin D. In embodiments, a fifth unit may include about 30 mg each, by protein weight, of peanut, soy, egg, milk, and a tree nut selected from the group consisting of almond, cashew, walnut, pecan, hazelnut, pistachio, or any combination thereof, and optionally Vitamin D.

Such contemplated kits may further include a sixth unit (e.g., a sixth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the sixth unit includes a different (e.g., additional) allergenic component from the fifth unit. In embodiments, a sixth unit may include about 30 mg, by protein weight, of a fish selected from the group consisting of salmon, cod, or any combination thereof, and optionally Vitamin D. In embodiments, a sixth unit may include about 30 mg each, by protein weight, of peanut, soy, egg, milk, a tree nut selected from the group consisting of almond, cashew, walnut, pecan, hazelnut, pistachio, or any combination thereof, and a fish selected from the group consisting of salmon, cod, or any combination thereof, and optionally Vitamin D.

Such contemplated kits may further include a seventh unit (e.g., a seventh allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the seventh unit includes a different (e.g., additional) allergenic component from the sixth unit. In embodiments, a seventh unit may include about 30 mg, by protein weight, of shrimp, and optionally Vitamin D. In embodiments, a seventh unit may include about 30 mg each, by protein weight, of peanut, soy, egg, milk, a tree nut selected from the group consisting of almond, cashew, walnut, pecan, hazelnut, pistachio, or any combination thereof, a fish selected from the group consisting of salmon, cod, or any combination thereof, and shrimp, and optionally Vitamin D.

Such contemplated kits may further include an eighth unit (e.g., an eighth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the eighth unit includes a different (e.g., additional) allergenic component from the seventh allergenic unit. In embodiments, an eighth unit may include about 30 mg, by protein weight, of sesame, and optionally Vitamin D. In embodiments, an eighth unit may include about 30 mg each, by protein weight, of peanut flour, soy, egg, milk, a tree nut selected from the group consisting of almond, cashew, walnut, pecan, hazelnut, pistachio, or any combination thereof, a fish selected from the group consisting of salmon, cod, or any combination thereof, shrimp, and sesame, and optionally Vitamin D.

Such contemplated kits may further include a ninth unit (e.g., a ninth allergenic unit comprising about 1 mg to about 1000 mg, e.g., about 2 mg to about 100 mg, about 5 mg to about 50 mg, about 15 mg, about 20 mg, about 25 mg, or about 30 mg by protein weight of an allergenic component selected from peanut, tree nut, cow's milk, soy, egg, fish and shellfish, and optionally Vitamin D), wherein the ninth unit includes a different (e.g., additional) allergenic component from the eighth unit. In embodiments, a ninth unit may include about 30 mg, by protein weight, of a non-nut plant selected from the group consisting of wheat, oat, or any combination thereof, and optionally Vitamin D. In embodiments, a ninth unit may include about 30 mg each, by protein weight, of peanut, soy, egg, milk, a tree nut selected from the group consisting of almond, cashew, walnut, pecan, hazelnut, pistachio, or any combination thereof, a fish selected from the group consisting of salmon, cod, or any combination thereof, shrimp, sesame, and a non-nut plant selected from the group consisting of wheat, oat, or any combination thereof, and optionally Vitamin D.

As used herein, an “allergenic unit” refers to a dosage form of an allergenic composition.

As used herein, an “allergenic composition” is understood to mean a composition that includes one or more different allergens or allergenic components. Allergenic compositions are understood to include “mixed allergen compositions” that include two or more different allergens, where any two given allergens are different if they are distinct from each other, e.g., they are compounds described by different chemical formula or compositions described by different components and/or amounts thereof The number of different allergens in a composition may vary, as desired. In certain embodiments, a mixed allergen composition comprises 2 or more different allergens, such as 3 or more different allergens, 4 or more different allergens, 5 or more different allergens, 6 or more different allergens, 7 or more different allergens, 8 or more different allergens, 9 or more different allergens, 10 or more different allergens, 15 or more different allergens, 20 or more different allergens, 25 or more different allergens, 30 or more different allergens, 40 or more different allergens, 50 or more different allergens, 75 or more different allergens, or 100 or more different allergens. In certain embodiments, a mixed allergen composition comprises 100 or fewer different allergens, such as 75 or fewer different allergens, 50 or fewer different allergens, 25 or fewer different allergens, 15 or fewer different allergens, or 10 or fewer different allergens. In certain embodiments, a composition may include 2 to 20 different allergens, 2 to 100 different allergens, or 2 to 1000 different allergens. In further embodiments, a composition may comprise 6 to 20 different allergens. In certain embodiments, a composition may consist essentially of 6 to 20 different protein allergens.

Allergens present in an allergenic composition (e.g., such as part of an allergenic unit) may vary, where in some instances an allergen present in the composition is one that induces an allergy in a susceptible subject. Allergens include any antigen, or active derivative thereof, that elicits a specific IgE response. Antigens include any substance that can stimulate the production of antibodies and can combine specifically with them. Allergens may have little or no intrinsic toxicity by themselves, but cause a pathological condition due to their ability to elicit an IgE-associated immune response, and, upon subsequent exposure, due to their ability to elicit IgE- and/or T cell-dependent hypersensitivity reactions. As such, an allergen includes any substance which is capable of stimulating a typical hypersensitivity reaction in atopic subjects. Allergens that may be present in a given allergenic composition include any substance found in a variety of different sources, e.g., foods, drugs, perfume, plants, the environment or biological systems (e.g., prokaryotic or eukaryotic cells or viruses), as well as chemical allergens.

It is appreciated that reference to an allergen or an allergenic composition (e.g., such as part of an allergenic unit or provided food product) may each include a plurality of different proteins as found in the naturally occurring allergen (either raw or cooked). For example, a provided food product may include a peanut allergen composition (which would include substantially all peanut proteins present in e.g., defatted peanuts, ground peanuts, etc.). As used herein the phrase “complete allergen” refers to all possible antigenic components of a given food product.

Allergens of interest include nut allergens. Nut allergens are allergens that include one or more compounds found in nuts, e.g., dry fruits that include an edible kernel or meat enclosed in a woody or leathery shell. Nut allergens of interest include, e.g. peanut allergens, (e.g., rAra h 1, rAra h 2, rAra h 3, rAra h 8 PR-10, rAra h 9 LTP, or peanut complete allergen), brazil nut allergens (e.g., rBer e 1, or brazil nut complete allergen), hazelnut or filbert allergens (e.g., rCor a 1 PR-10, rCor a 8 LTP, nCor a 9, rCor a 14, or hazel nut complete allergen), walnut allergens (e.g., rJug r 1, rJug r 3 LTP, or walnut complete allergen), cashew allergens (e.g., cashew component allergens, or cashew complete allergen), pistachio allergens (e.g., pistachio component allergens, or pistachio complete allergen), pecan allergens (e.g., pecan component allergens, or pecan complete allergen), almond allergens (e.g., almond component allergens, or almond complete allergen), or tree nut component package allergens (e.g., one or more allergens from e.g., cashew nut, walnut, hazelnut, or brazil nut).

Allergens of interest include animal allergens. Animal allergens are allergens that include one or more compounds found in animals, including both vertebrates and invertebrates. Vertebrate animal allergens that may be present in an allergenic composition include avian allergens (e.g., egg allergens, e.g., nGal d 1 Ovomucoid, n Gal d 2 Ovalbumin, nGal d 3 Conalbumin, or egg white complete allergen), mammalian allergens (e.g. milk allergens, e.g., nBos d 4 alpha-lactalbumin, nBos d 5 beta-lactoglobulin, nBos d 8 Casein, nBos d Lactoferrin, or milk complete allergen), or fish allergens (e.g., rCyp c 1, rGad c 1, cod complete allergen, white fish allergens, or pink fish allergens). Invertebrate animal allergens that may be present in an allergenic composition include crustacean allergens (e.g., shrimp allergens, e.g., rPen a 1 tropomyosin, or shrimp complete allergen), or insect allergens (e.g., bee sting venom allergen, wasp sting venom allergen, or mosquito bite allergen).

Allergens of interest include non-nut plant allergens, i.e., plant allergens that are not nut allergens. Plant allergens are allergens that include one or more compounds found in plants. Plant allergens of interest include wheat allergens (e.g., rTri a 19 Omega-5 Gliadin, gliadin wheat, rTri a 14 LTP, or wheat complete allergen), fruit allergens (e.g., kiwi allergens, e.g., rAct d 8 PR-10, or kiwi complete allergen), vegetable allergens (e.g., carrot allergens, or celery allergens, e.g., rApi g 1.01 PR-10, rPhl p 12, or celery complete allergen), CCD MUXF3 from Bromelain, legume allergens (e.g., soy allergens or chickpea allergens, e.g., rGly m 4 PR-10, nGly m 5 Beta-conglycinin, nGly m 6 Glycinin, or soy complete allergen), stone fruit allergens, e.g., f419, f420, f421, f95, f242, o214 rPru p 1 PR-10, rPru p 3 LTP, or stone fruit primary complete allergen), oat allergens (e.g., oat component allergens, or oat complete allergen), or seed allergens (e.g., sesame allergens, e.g., sesame seed component allergens, or sesame see complete allergen).

Additional types of allergens that may be present in an allergenic unit, component or composition include, e.g., non-food animal allergens (e.g., cats or dog fur and dander, cockroach calyx, dust mite excretion), drug allergens (penicillin, sulfonamides, salicylates, local anesthetics), mold spore allergens, latex allergens, metal allergens, or plant pollen allergens (e.g. from grass, e.g., ryegrass or timothy-grass, from weeds, e.g., ragweed, plantago, nettle, Artemisia, vulgaris, chenopodium album, sorrel, or e.g., from trees, e.g., birch alder, hazel, hornbeam, aesculus, willow, poplar, platanus, tilia, or olea).

In certain embodiments, an allergenic unit, component or composition may comprise one, two, or more allergens selected from a group consisting of cashew, pistachio, walnut, pecan, white fish, pink fish, shrimp, peanut, soy, hazelnut, almond, milk, egg, crab, wheat, and sesame.

In certain embodiments, an allergenic unit, component, or composition may comprise one, two, or more allergens selected from a group consisting of peanut, soy, almond, cashew, hazelnut, pecan, pistachio, walnut, wheat, oat, milk, egg, cod, salmon, shrimp, and sesame.

The amount of a given allergen in an allergenic unit, component, or may vary, as desired. In certain embodiments, the amount of a given allergen ranges from about 1 to about 15,000 mg, about 5 to about 15,000 mg, about 10 to about 10,000 mg, about 15 to about 5,000 mg, about 10 to about 100 mg, or about 15 to about 100 mg. In certain embodiments, the amount of a given allergen is about 30 mg. The weight percentage of a given allergen in an allergenic unit, component, or may vary, as desired. In certain embodiments, the weight percentage of a given allergen in an allergenic unit, component, or composition ranges from about 0.1 to about 99.9 wt. %, about 0.1 to about 15 wt. %, about 0.1 to about 99.9 wt. %, about 15 to about 99.9 wt. %, or about 25 to about 65 wt. %. The amount of a given allergen in an allergenic unit, component, or composition may be recited by total mass, or by protein mass, which may vary for a given allergen depending upon the weight percentage of protein in that allergen.

In certain embodiments, if more than one allergen is present in an allergenic unit, component, or composition, e.g., in a mixed allergen composition, any two of the mixed allergens, or all of the mixed allergens, are present in equal parts, e.g., in a 1:1 ratio, such that each allergen is present in the composition in equal weight.

A disclosed allergenic unit, component, or composition may include one or more vitamins, as desired. Vitamins that may be present include. e.g., vitamin A (e.g., in an amount ranging from 1 to 35,000 IU), vitamin C (e.g., in an amount ranging from about 1 to about 1,000 mg), vitamin D (e.g., in an amount ranging from about 1 to about 4,000 IU, i.e., from about 0.025 to about 100 mcg), vitamin E (e.g., in an amount ranging from about 1 to about 450 IU) vitamin K (e.g., in an amount ranging from about 1 to about 250 mcg), vitamin B-1 (thiamin; e.g., in amount ranging from about 1 to about 15 mg), vitamin B-2 (riboflavin; e.g., in an amount ranging from about 1 to about 17 mg) vitamin B-3 (niacin; e.g., in an amount ranging from about 1 to about 200 mg), vitamin B-5 (pantothenic acid; e.g., in an amount ranging from about 1 to about 100 mg), vitamin B-6 (pyridoxine; e.g., in an amount ranging from about 1 to about 30 mg) vitamin B-9 (folic acid; e.g., in an amount ranging from about 1 to about 4,000 mcg), vitamin B-12 (cobalamin; e.g., in an amount ranging from about 1 to about 250 mcg), vitamin H (biotin; e.g., in an amount ranging from about 1 to about 1,000 mcg) and combinations thereof In certain embodiments, an allergenic unit, component, or composition comprises vitamin D. In certain embodiments, an allergenic unit, component, or composition comprises about 400 IU, i.e., about 10 mcg, of vitamin D.

For example, a contemplated method comprises administering to the subject two or more allergenic units, wherein a first allergenic unit is administered daily and each additional allergenic unit is administered sequentially; wherein each allergenic unit is selected from the group consisting of: an allergenic unit comprising about 30 mg of peanut allergen and optionally Vitamin D; an allergenic unit comprising about 30 mg of soy allergen; an allergenic unit comprising about 30 mg of egg allergen; an allergenic unit comprising about 30 mg of milk allergen; an allergenic unit comprising: about 30 mg each of at least one tree nut allergen selected from the group consisting of almond allergen, cashew allergen, walnut allergen, pecan allergen, hazelnut allergen, and pistachio allergen; an allergenic unit comprising: about 30 mg each of salmon allergen, and cod allergen; an allergenic unit comprising: about 30 mg of shrimp allergen; an allergenic unit comprising about 30 mg of sesame allergen; and an allergenic unit comprising at least one of about 30 mg each of wheat and oat allergens; wherein each selected allergenic unit is administered after at least two days of administration of a previously selected allergenic unit.

For example, a contemplated method comprises administering to a subject a first allergenic unit comprising about 30 mg of peanut allergen and optionally Vitamin D; a second allergenic unit comprising about 30 mg of soy allergen, wherein the second allergenic unit is administered at least about 2 days after the first allergenic unit is first administered, a third allergenic unit comprising about 30 mg of egg allergen, wherein the third allergenic unit is administered at least about 2 days after the second allergenic unit is first administered. a fourth allergenic unit comprising about 30 mg of milk allergen, wherein the fourth allergenic unit is administered at least about 2 days after the third allergenic unit is first administered; a fifth allergenic unit comprising: about 30 mg each of almond allergen, cashew allergen, walnut allergen, pecan allergen, hazelnut allergen, and pistachio allergen, wherein the fifth allergenic unit is administered at least about 2 days after the fourth allergenic unit is first administered; a sixth allergenic unit comprising: about 30 mg each of salmon allergen, and cod allergen, wherein the sixth allergenic unit is administered at least about 2 days after the fifth allergenic unit is first administered; a seventh allergenic unit comprising: about 30 mg of shrimp allergen, wherein the seventh allergenic unit is administered at least about 2 days after the sixth allergenic unit is first administered, an eighth allergenic unit comprising: about 30 mg of sesame allergen, wherein the eighth allergenic unit is administered at least about 2 days after the seventh allergenic unit is first administered; a ninth allergenic unit comprising: about 30 mg each of wheat and oat allergens, wherein the ninth allergenic unit is administered at least about 2 days after the eighth allergenic unit is first administered.

It is appreciated that a disclosed allergen or protein may be in the form of a flour, powder, meal, paste, etc., In some embodiments, a disclosed unit or composition comprises about 30 mg protein by weight of each specific protein or allergen contained therein, e.g. about 30 mg by protein weight of an allergenic component each selected as described herein from peanut, tree nut, cow's milk, soy, egg, fish and shellfish.

Also provided are physiological acceptable units, components or compositions that include a disclosed allergenic composition and a physiologically acceptable delivery vehicle. Disclosed allergenic units, components, or compositions can be incorporated into a variety of formulations for administration to a subject. More particularly, a disclosed allergenic unit, component, or composition can be formulated into a physiological acceptable composition by combination with appropriate, physiologically acceptable carriers or diluents, for example, a vegetable oil. In certain embodiments, a disclosed allergenic unit, component, or composition is designed for oral administration, for example, as foods, tablets, troches, lozenges, aqueous or oily suspensions, dispersible powders or granules, emulsions, hard or soft capsules, or syrups or elixirs, gums, etc. Compositions intended for oral use may be prepared according to any convenient protocol for the manufacture of pharmaceutical compositions and such compositions may contain one or more agents selected from the group consisting of sweetening agents, flavoring agents, coloring agents and preserving agents in order to provide palatable preparations.

Also provided are allergenic units, components, or compositions that are food products. Food products of interest include a disclosed allergenic unit, component, or composition in combination with a food delivery vehicle. By food delivery vehicle is meant a delivery vehicle that is a nourishing substance that is eaten, drunk, or otherwise taken into the body to sustain life, provide energy, promote growth, etc. Examples of food delivery vehicles or food products of interest include, but are not limited to: baby or infant formula, baby food (e.g., pureed food suitable for infant or toddler consumption), chips, cookies, breads, spreads, creams, yogurts, liquid drinks, chocolate containing products, candies, ice creams, cereals, coffees, pureed food products, etc. In certain embodiments, the composition is a food supplement.

In certain embodiments, a disclosed allergenic unit, component or composition is in a liquid form. In certain embodiments, a liquid allergenic unit, component, or composition may include a bulking agent. Exemplary bulking agents include maltodextrin, sucrose, trehalose, trehalose dihydrate, mannitol, lactose, or raffinose or any combination thereof. In certain embodiments, the bulking agent comprises maltodextrin, or sucrose, or a combination thereof In certain embodiments, the bulking agent comprises maltodextrin and sucrose at a weight ratio of about 3:1. In certain embodiments, an allergenic unit, component, or composition may include excipients, e.g., a food safe oil, a polysaccharide (e.g., gellan gum), flavoring, and a food safe salt (e.g., dipotassium phosphate).

In certain embodiments an allergenic unit, component, or composition is an aqueous suspension containing a disclosed allergenic component in admixture with excipients suitable for the manufacture of aqueous suspensions. Such excipients may include suspending agents, for example sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

In certain embodiments an allergenic unit, component, or composition is an oily suspension containing an allergenic composition suspended in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Disclosed allergenic units, components, or compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, preservative and flavoring and coloring agents. A disclosed composition may be in the form of a sterile aqueous or oleagenous suspension. This suspension may be formulated according to the known art using those suitable dispersing or wetting agents and suspending agents which have been mentioned above. The sterile preparation may also be a sterile solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Throughout the description, where apparatus, devices, and systems are described as having, including, or comprising specific components, or where processes and methods are described as having, including, or comprising specific steps, it is contemplated that, additionally, there are apparatus, devices, and systems that consist essentially of, or consist of, the recited components, and that there are processes and methods that consist essentially of, or consist of, the recited processing steps.

The foregoing examples are presented herein for illustrative purposes only, and should not be construed as limiting in any way.

### EXAMPLES

### Example 1

This example describes an exemplary method for the introduction of allergens to a subject.

Children aged 4 months to 5 years will be administered an oral allergenic unit including one or more allergenic components daily over the course of a year. Over the course of the first 10 weeks, the allergenic unit will be modified to introduce one or more additional allergenic components each week, as depicted in Table 1.

TABLE 1

| Week(s) | Allergenic Unit |
|---|---|
| 1 | Unit A (30 mg by protein weight peanut flour and |
| | 400 IU vitamin D) |
| 2 | Unit B (Unit A + 30 mg by protein weight soy |
| | protein isolate) |
| 3 | Unit C (Unit B + 30 mg by protein weight dried |
| | whole eggs) |
| 4 | Unit D (Unit C + 30 mg by protein weight milk |
| | protein isolate) |
| 5 | Unit F (Unit E + 30 mg by protein weight each of |
| | blanched almond flour, cashew flour, walnut flour, |
| | pecan meal, walnut flour, and pistachio meal) |
| 6 | Unit G (Unit F + 30 mg by protein weight each of |
| | salmon powder and codfish powder) |
| 7 | Unit H (Unit G + 30 mg by protein weight shrimp |
| | powder) |
| 8 | Unit I (Unit H + 30 mg by protein weight sesame |
| | seed flour) |
| 9-52 | Unit J (Unit I + 30 mg by protein weight each wheat |
| | gluten powder and oat) |

Allergenic units will be prepared as follows. In a dry mill, the allergenic ingredients along with bulking ingredients to lower moisture and fat content to acceptable levels as needed (maltodextrin and sugar) will be dry milled to achieve desired particle size. Next, the dry milled allergenic ingredient mix will be incorporated into liquid format by shear mixing into lukewarm water to solubilize and suspend the solids, and the product will be held in a tank for 1 hour with constant agitation to hydrate the proteins. Next, the shear mixed product will be wet milled/homogenized to reduce particle size in the liquid suspension or slurry, with recirculation of the product multiple times to decrease sedimentation and improve opacity. Following homogenization, the remaining ingredients will be incorporated by shear mixing in the fat, buffer, and gellan gum to improve stability, visual appearance and the suspension of the solids. Flavor will be added, if needed. Next, an ultra-high temperature unit will be used for ultra-high temperature processing for 15 seconds at 287° F. for a thermal lethality step to ensure food product safety. The resulting product will further homogenized with an in-line homogenizer to produce a consistent range of particle sizes for solid components and fat globules, and to uniformly distribute particles throughout the liquid matrix. Lastly, the composition will be aseptically filled into single-serve packing for storage at ambient temperature.

Children will be monitored for allergies as follows. Peripheral blood mononuclear cells (PBMCs) and plasma will be extracted from blood via ficoll procedure and stored in liquid nitrogen and at ˜80° C., respectively. Allergen-specific IgE and IgG4 will be measured using a standard ImmunoCAP assay (Phadia, Uppsala, Sweden). Increased specific IgE is indicative of an inflammatory allergic state, and increased IgG4 levels are indicative of a protective anti-inflammatory state. PBMCs from subjects will be labeled with carboxyfluorescein diacetate succinimidyl ester (CFSE) and cultured with food allergens at 100 μg/mL or anti-CD3/CD28 (to test for nonspecific proliferation capacity) for 7 days to identify T cell subsets. At day 7, cells will be washed and stained for surface CD4, CD25, CD127, CD45RO, CD45RA, CD40L, and CD69 and intracellular Foxp3 and IL-10 along with Live/Dead staining (Invitrogen). Th2 cells will be defined as the cells that proliferated in response to food allergen (CFSElo) and were CD41 IL-4, IL-13 cells. Antigen-induced T cells will also be identified by isolating CD40L and CD69 double-positive cells after antigen stimulation. Lower degrees of Th2 proliferation on exposure to allergens will indicate decreases in allergic response.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A method for introducing allergens to a human subject for the treatment or prevention of food allergies, the method comprising administering to the subject two or more allergenic units, wherein the initial administration of each allergenic unit is staggered, each allergenic unit is administered daily, every other day, every 2 or 3 days, or weekly, the initial administration of each additional allergenic unit is at least two days after the initial administration of a previously administered allergenic unit, and each previously administered allergenic unit continues to be administered in addition to each additional allergenic unit as each additional allergenic unit is administered; and
wherein each allergenic unit comprises a different food allergen selected from the group consisting of: an allergenic unit comprising least one tree nut selected from the group consisting of almond, cashew, walnut, pecan, hazelnut, and pistachio; an allergenic unit comprising a white fish and/or a pink fish; and an allergenic unit comprising shellfish.

2. A method for introducing allergens to a human subject comprising administering to the subject:
a first allergenic unit comprising about 30 mg of peanut protein and optionally Vitamin D; wherein the first allergenic unit is administered daily, every other day, every 2 or 3 days, or weekly.

3. The method of claim 2, further comprising administering to the subject a second allergenic unit comprising about 30 mg of soy protein, wherein the second allergenic unit is administered at least about 2 days after the first allergenic unit is first administered, and wherein the second allergenic unit is administered daily, every other day, every 2 or 3 days or weekly.

4. The method of claim 3, further comprising administering to the subject a third allergenic unit comprising about 30 mg of egg protein, wherein the third allergenic unit is administered at least about 2 days after the second allergenic unit is first administered, and wherein the third allergenic unit is administered daily, every other day, every 2 or 3 days or weekly.

5. The method of claim 4, further comprising administering to the subject a fourth allergenic unit comprising about 30 mg of milk protein, wherein the fourth allergenic unit is administered at least about 2 days after the third allergenic unit is first administered, and wherein the fourth allergenic unit is administered daily, every other day, every 2 or 3 days or weekly.

6. The method of claim 5, further comprising administering to the subject a fifth allergenic unit comprising about 30 mg each of almond protein, cashew protein, walnut protein, pecan protein, hazelnut protein and pistachio protein, wherein the fifth allergenic unit is administered at least about 2 days after the fourth allergenic unit is first administered, and wherein the fifth allergenic unit is administered daily, every other day, every 2 or 3 days or weekly.

7. The method of claim 6, further comprising administering to the subject a sixth allergenic unit comprising about 30 mg each of salmon protein and cod protein, wherein the sixth allergenic unit is administered at least about 2 days after the fifth allergenic unit is first administered, and wherein the sixth allergenic unit is administered daily, every other day, every 2 or 3 days or weekly.

8. The method of claim 7, further comprising administering to the subject a seventh allergenic unit comprising about 30 mg of shrimp protein, wherein the seventh allergenic unit is administered at least about 2 days after the sixth allergenic unit is first administered, and wherein the seventh allergenic unit is administered daily, every other day, every 2 or 3 days or weekly.

9. The method of claim 8, further comprising administering to the subject an eighth allergenic unit comprising about 30 mg of sesame protein, wherein the eighth allergenic unit is administered at least about 2 days after the seventh allergenic unit is first administered, and wherein the eighth allergenic unit is administered daily, every other day, every 2 or 3 days or weekly.

10. The method of claim 9, further comprising administering to the subject a ninth allergenic unit comprising about 30 mg each of wheat protein and oat protein, wherein the ninth allergenic unit is administered at least about 2 days after the eighth allergenic unit is first administered, and wherein the ninth allergenic unit is administered daily, every other day, every 2 or 3 days or weekly.

11. The method of claim 5, further comprising administering to the subject a fifth allergenic unit comprising about 30 mg each of 1-6 tree nut proteins, wherein the fifth allergenic unit is administered at least about 2 days after the fourth allergenic unit is first administered, and wherein the fifth allergenic unit is administered daily, every other day, every 2 or 3 days or weekly.

12. The method of claim 6, further comprising administering to the subject a sixth allergenic unit comprising about 30 mg each of one, two or three fish proteins, wherein the sixth allergenic unit is administered at least about 2 days after the fifth allergenic unit is first administered, and wherein the sixth allergenic unit is administered daily, every other day, every 2 or 3 days or weekly.

13. The method of claim 7, further comprising administering to the subject a seventh allergenic unit comprising about 30 mg of a shellfish protein, wherein the seventh allergenic unit is administered at least about 2 days after the sixth allergenic unit is first administered, and wherein the seventh allergenic unit is administered daily, every other day, every 2 or 3 days or weekly.

14. The method of claim 8, further comprising administering to the subject an eighth allergenic unit comprising about 30 mg of sesame protein, wherein the eighth allergenic unit is administered at least about 2 days after the seventh allergenic unit is first administered, and wherein the eighth allergenic unit is administered daily, every other day, every 2 or 3 days or weekly.

15. The method of claim 9, further comprising administering to the subject a ninth allergenic unit comprising about 30 mg each of wheat protein and oat protein, wherein the ninth allergenic unit is administered at least about 2 days after the eighth allergenic unit is first administered, and wherein the ninth allergenic unit is administered daily, every other day, every 2 or 3 days or weekly.

16. A method for introducing allergens to a human subject comprising administering to the subject:
a first allergenic unit comprising about 30 mg of peanut protein and optionally Vitamin D;
a second allergenic unit comprising about 30 mg of soy protein, wherein the second allergenic unit is administered at least about 2 days after the first allergenic unit is first administered,
a third allergenic unit comprising about 30 mg of egg protein, wherein the third allergenic unit is administered at least about 2 days after the second allergenic unit is first administered.
a fourth allergenic unit comprising about 30 mg of milk protein, wherein the fourth allergenic unit is administered at least about 2 days after the third allergenic unit is first administered;
a fifth allergenic unit comprising about 30 mg each of almond protein, cashew protein, walnut protein, pecan protein, hazelnut protein and pistachio protein, wherein the fifth allergenic unit is administered at least about 2 days after the fourth allergenic unit is first administered;
a sixth allergenic unit comprising about 30 mg each of salmon protein and cod protein, wherein the sixth allergenic unit is administered at least about 2 days after the fifth allergenic unit is first administered;
a seventh allergenic unit comprising about 30 mg of shrimp protein, wherein the seventh allergenic unit is administered at least about 2 days after the sixth allergenic unit is first administered,
an eighth allergenic unit comprising about 30 mg of sesame protein, wherein the eighth allergenic unit is administered at least about 2 days after the seventh allergenic unit is first administered; and
a ninth allergenic unit comprising about 30 mg each of wheat protein and oat protein, wherein the ninth allergenic unit is administered at least about 2 days after the eighth allergenic unit is first administered.

17. A method for introducing allergens to a human subject comprising administering to the subject two or more allergenic units, wherein a first allergenic unit is administered daily and each additional allergenic unit is administered sequentially; wherein each allergenic unit is selected from the group consisting of:
an allergenic unit comprising about 30 mg by protein weight of peanut and optionally Vitamin D;
an allergenic unit comprising about 30 mg by protein weight of soy;
an allergenic unit comprising about 30 mg by protein weight of egg;
an allergenic unit comprising about 30 mg by protein weight of milk;
an allergenic unit comprising about 30 mg by protein weight each of at least one tree nut selected from the group consisting of almond, cashew, walnut, pecan, hazelnut, and pistachio;
an allergenic unit comprising about 30 mg by protein weight each of salmon and cod;
an allergenic unit comprising about 30 mg by protein weight of shrimp;
an allergenic unit comprising about 30 mg by protein weight of sesame; and
an allergenic unit comprising about 30 mg by protein weight of at least one of wheat and oat;
wherein each selected allergenic unit is administered after at least two days of administration of a previously selected allergenic unit.

18. A kit comprising:
a first unit comprising one allergenic component selected from the group consisting of: peanut, tree nut, cow's milk, soy, egg, fish and shellfish; and optionally Vitamin D;
a second unit comprising a first subunit and a second subunit, wherein the first subunit comprises the same allergenic component as the first unit, and the second subunit comprises one allergenic component selected from the group consisting of: peanut, tree nut, cow's milk, soy, egg, fish and shellfish; and optionally Vitamin D, wherein the second subunit allergenic component is different than the first subunit allergenic component; and wherein the first subunit and second subunit may be separate or combined.

19. The kit of claim 18, further comprising one or more sequential additional units, where each additional unit comprises a first additional subunit and a second additional subunit, wherein the first additional subunit comprises the same allergenic components as the previous unit, and the second additional subunit comprises one allergenic component selected from the group consisting of: peanut, tree nut, cow's milk, soy, egg, fish and shellfish; and optionally Vitamin D, wherein the second additional subunit allergenic component is different than any of the first additional subunit allergenic components; and wherein the first additional subunit and second additional subunit may be separate or combined.

20. The kit of claim 18 or 19, wherein the allergenic components are each about 30 mg protein.
